(19) United States
(12) Patent Application Publication
(10) Pub. No.: US 2017/0216343 A1
(43) Pub. Date: Aug. 3, 2017

(54) VISCOSUPPLEMENT COMPOSITION COMPRISING ULVAN FOR TREATING ARTHRITIS

(71) Applicant: Stemmatters, Biotecnologia e Medicina Regenerativa SA, Barco GMR (PT)

(72) Inventors: Rui Pedro Romero Amandi de SOUSA, Barco GMR (PT); Ana Catarina Freire GERTRUDES, Barco GMR (PT); Cristina CORREIA, Barco GMR (PT); Alain José da Silva MORAIS, Barco GMR (PT); Cristiana da Mota Martins GONÇALVES, Barco GMR (PT); Hajer RADHOUANI, Barco GMR (PT); Carlos Alberto Vilela GOMES, Barco GMR (PT); Tírcia Susete Xavier Carlos dos SANTOS, Barco GMR (PT); Joaquim Miguel Antunes Correia de OLIVEIRA, Barco GMR (PT); João Duarte Coelho do Sameiro ESPREGUEIRA-MENDES, Barco GMR (PT); Rui Luís Gonçalves dos REIS, Barco GMR (PT)

(21) Appl. No.: 15/311,779

(22) PCT Filed: May 18, 2015
(86) PCT No.: PCT/PT2015/000023

§ 371 Date: Nov. 16, 2016

(30) Foreign Application Priority Data
May 16, 2014 (GB) 1408752.2

(51) Int. Cl. A61K 31/737; A61K 9/00; A61K 31/728

(52) U.S. Cl. CPC A61K 31/737; A61K 31/728; A61K 9/0019

(57) ABSTRACT

There is described an ulvan containing composition. This composition is a viscosupplement composition and can be used in the treatment or prophylaxis of arthritis. Also described is a method of treating a musculoskeletal disease, such as 5 arthritis, by administering the ulvan containing composition.

### FIELD OF THE INVENTION

The present invention relates to an ulvan containing composition. This composition is a viscosupplement composition and can be used in the treatment of arthritis. The present invention also relates to a method of treating arthritis by administering the ulvan containing composition.

### BACKGROUND TO THE INVENTION

Arthritis is a chronic inflammation of the joint, which is normally accompanied by pain, swelling of surrounding connective tissue and limitation of movement. The most prevalent forms of arthritis are osteoarthritis and rheumatoid arthritis, both of which are progressive, degenerative diseases leading to varying degrees of disability. As a result of these diseases, cartilage and bone of the joint undergo progressive destruction, followed by loss of mobility and increased pain.

Osteoarthritis (OA), also designated as degenerative joint disease or arthrosis, is among the most common musculoskeletal disorders. Symptoms of OA are pain, swelling and stiffness of the articulation. Onset of QA is likely to be initiated many years prior to its clinical diagnosis, and it persists until the end stage of the disease where almost all the articular cartilage of the affected joints is lost.

Osteoarthritis is characterized by breakdown of articular cartilage due to biological or mechanical factors, which changes normal load transmission in the joint and produces pain. Inflammation of synovium may additionally cause pain, which may be triggered by cartilage particles (meniscal or articular). Cartilage degeneration stimulates production of extracellular matrix degrading enzymes and produces inflammatory mediators in the joint, which will further contribute to inflammation of synovium.

OA affects predominantly articular cartilage, which degrades by gradual loss of its extracellular matrix (ECM) composed mainly of aggrecan and type II collagen. Decrease in proteoglycan aggrecan content occurs first, resulting in a concomitant decrease in compressive stiffness. This is followed by damage to the collagen fibrillar network which is the main component responsible for tensile properties of cartilage.

Aggrecan degradation is associated with upregulation of aggrecanases, a disintegrin and metalloprotease with thrombospondin motifs (ADAMTS-) 4 and 5 as well as matrix metalloproteinases (MMPs). Excessive cleavage of type II collagen is caused by upregulation of synthesis and activity of metalloproteinases, in particular MMP-13.

Rheumatoid arthritis is an inflammatory condition with widespread synovial joint involvement. It is the most common form of chronic polyarthritis, and although it is a systemic disease, it predominantly affects peripheral joints. Persistent synovitis leads to joint destruction, which results in long-term morbidity and increased mortality. Its etiology remains unknown. Modern treatment is ineffective at controlling disease activity and reducing long-term disability, and early treatment aimed at controlling disease activity is the present prevention strategy.

Viscosupplementation consists of the injection of a gel-like, polymeric substance into the joint. The biocompatible lubricant aims at lubricating the cartilage interface, thus reducing pain and improving joint flexibility. This method of treatment usually requires several injections, as benefits are mostly temporary. Currently used substances degrade within weeks to months due to the action of enzymes in the synovial fluid which break down the polymer structure. Substances used in viscosupplementation include hyaluronic acid (HA) and poly-sulphated glycosaminoglycans (PSGAGS).

Hyaluronic acid (HA) is a natural complex sugar of the glycosaminoglycan (GAG) family. HA is a long-chain polymer containing disaccharide units of glucuronic acid and N-acetylglucosamine. HA is a naturally occurring substance found in the synovial (joint) fluid, where it acts as a lubricant and shock absorber during joint functioning. Intra-articular (IA) injections of HA have been clinically used to manage OA in patients with questionable success.

Hyaluronic acid has been widely studied for osteoarthritis application, either alone or mixed with other compounds. However, the therapeutic effect of hyaluronic acid is relatively short-lasting due to the action of the native hyaluronidase enzyme, which extensively degrades the polymer chain, leading to limited clinical efficacy. Accordingly, multiple injections of hyaluronic acid are required over time in order to maintain acceptable levels of intact hyaluronic acid at the site of action for sustained pain relief. This administration regime presents added costs and inconvenience for both the patient and physician.

Evidence from clinical studies suggests that the chondroprotective effect of HA is due to significant reduction in joint space narrowing, inhibition of matrix metalloproteinases (MMPs) in synovial tissue and reduction of MMP-9 in the synovial fluid in HA-treated patients suffering from OA.

Polysaccharides with high sulphate content are generally described as sulphated polysaccharides (SPS). SPS have gained interest in medicine due to their bioactivity and physico-chemical performance, SPS have been extensively studied for their antiviral, antitumor, antiangiogenic, anticoagulant, antioxidant, anti-inflammatory, antiproliferative, antiparasitic, antimetastatic, and immunomodulating activities.

SPS can be classified as natural (extracted from plants, animals and microorganisms) or synthetic (chemically modified or synthesized). Most mammalian SPS are GAGs, being a part of the extracellular matrix of tissues. Bioactivity of SPS depends upon the molecular weight, degree of sulphation, position of sulphate group and solubility in water.

Several SPS have been proposed as viscosupplements for the treatment of osteoarthritis. Chondroitin sulphate (CS) is a sulphated GAG composed of a chain of alternating sugars (N-acetylgalactosamine and glucuronic acid). Experimental studies have demonstrated anti-inflammatory, anabolic and anti-catabolic properties. Sulphation appears to influence the therapeutic properties of CS in osteoarthritis. CS has been referred as effective in reducing progression of joint space narrowing in patients with OA.

SPS have a high application potential in the context of OA. The intrinsic bioactive performance of SPS coupled with improved stability against enzymatic degradation compared to HA, makes these polymers potentially suitable for viscosupplement compositions. Although the use of SPS in the context of OA treatment has potential merits due to their wide bioactive spectrum and intrinsic physicochemical properties, the development of improved, longer-acting viscosupplement compositions is very challenging for those skilled in the art.

In a review by Lahaye et al., (2007), it is recited that ulvan may serve as a source of rare sugars for synthesis of fine chemicals and heparin analogues. Ulvan is also suggested to be of pharmaceutical interest due to its anti-tumour, antiviral and immune modulation activities amongst others, as well as its ability to reduce serum cholesterol and triglyceride levels. Potential applications in the arthritis setting are not discussed.

Japanese patent application JP2009057285 claims the use of an angiogenesis inhibitor comprising a sulphated polysaccharide comprising rhamnose as the main component of a constituent monosaccharide or a salt thereof for the treatment of generalised disorders such as solid tumours, vascular lesions, skin, bone and joint, genital, eye and lung diseases. Such sulphated polysaccharides may be obtained from several seaweed species, including the green sea lettuce Ulva. In order to produce the technical effect, the sulphated polysaccharides must function as an angiogenesis inhibitor. The usefulness of ulvan as a viscosupplement composition acting via angiogenesis inhibition in the arthritis setting is not mentioned.

### SUMMARY OF THE INVENTION

The development of compositions for OA treatment that incorporate SPS demands the selection of an SPS taking into account many molecular parameters such as chemical structure, sulphation degree and molecular weight, among others.

The present invention addresses the need for compositions for treatment and prophylaxis of arthritis in animals, preferably in humans, that reduce the number of administrations and the respective dose per administration. The present invention relates to alternative viscosupplement compositions based on ulvan. Ulvan has not been previously used as a viscosupplement, nor has it been used in the treatment or prophylaxis of arthritis, in particular, osteoarthritis.

Therefore, in a first aspect, the present invention provides a composition for the treatment or prophylaxis of arthritis, the composition comprising ulvan. Thus, the composition is a viscosupplement composition.

Ulvan based viscosupplements benefit from the bioactive properties of ulvan and exhibit improved degradation behaviour as compared to existing viscosupplement compositions such as hyaluronic acid.

Ulvan is a sulphated polysaccharide composed of rhamnose, xylose and uronic acids, namely glucuronic acid and iduronic acid. Ulvan contains a repeating disaccharide unit consisting of two monomers selected from rhamnose, xylose, glucuronic acid and iduronic acid. Ulvan repeating disaccharide units include at least:

(i) [glucuronic acid-rhamnose sulphate];
(ii) [iduronic acid-rhamnose sulphate];
(iii) [xylose-rhamnose sulphate]; and
(iv) [xylose sulphate-rhamnose sulphate].

Therefore, in some embodiments, the ulvan is a polymer of one or more of the above disaccharide units.

In particular, the ulvan repeating disaccharide units include:

(i) type A3S [>4-beta-D-glucuronic acid-(>4)-alpha-L-rhamnose 3-sulphate-1>];
(ii) type B3S [>4-alpha-L-iduronic acid-(>4)-alpha-L-rhamnose 3-sulphate-1>];
(iii) type U3S [>4-beta-D-xylose-(1>4)-alpha-L-rhamnose 3-sulphate-1>];
(iv) type U2′S3S [>4-beta-D-xylose 2-sulphate-(1>4)-alpha-L-rhamnose 3-sulphate-1>].

Therefore, in some embodiments, the ulvan is a polymer of one or more of the above disaccharide units.

The ulvan may be associated with salts that act as molecule stabilisers.

The ulvan used in the present invention can come from any suitable source. Ulvan can be extracted from vegetal species, for example green algae, for example as described by Alves et al., 2010, although other methods for its production such as bacterial fermentation or organic synthesis can also be used.

Some or all of the monomers in ulvan are sulphated. For example, ulvan may comprise one or more of xylose sulphate, rhamnose sulphate, sulphated glucuronic acid and sulphated iduronic acid.

The sulphate content of naturally occurring ulvan varies typically between 0.1% and 25%. However, this can be increased by the partial or total sulphation of free hydroxyl groups in the ulvan, which gives rise to the respective desired sulphation degree.

In some embodiments, the sulphation degree of ulvan is 0.1% to 75%. This means that 0.1% to 75% of the hydroxyl groups of rhamnose, xylose, glucuronic acid and iduronic acid are sulphated. Preferably, the sulphation degree of ulvan is 0.1% to 40%. More preferably, the sulphation degree of ulvan is 0.1% to 30%. Even more preferably, the sulphation degree of ulvan is 0.1% to 20%. Most preferably, the sulphation degree of ulvan is 0.1% to 6%. The ulvan sulphation degree can be adjusted by chemical modification according to methods known to those skilled in the art (Papy-Garcia et al., (2005) and Nagasawa et al., (1977)).

In some embodiments, the ulvan has an average molecular weight between 100 and 10,000 kDa. Preferably, the ulvan has an average molecular weight between 100 and 5,000 kDa. More preferably, the ulvan has an average molecular weight between 100 and 3,000 kDa. Even more preferably, the ulvan has an average molecular weight between 100 and 1,200 kDa. Most preferably, the ulvan has an average molecular weight between 100 and 600 kDa

Naturally occurring ulvan has a relatively high average molecular weight. If the molecular weight is regarded as too high, lower molecular weight molecules like oligosaccharides can be derived from ulvan by hydrolysis of the starting molecule. Preferably, the lower molecular weight molecules derived from ulvan are in the range of 30 to 100 kDa. If necessary, the lower molecular weight ulvan molecules can be covalently crosslinked to increase the molecular weight. Preferably, the crosslinked ulvan molecules have an average molecular weight between 100 and 10,000 kDa.

Adjustment of the degree of sulphation of ulvan can be performed before and/or after the hydrolysis step. In some embodiments, the oligosaccharides produced by the hydrolysis have a molecular weight below 100 kDa.

In some embodiments, the ulvan comprises 5% to 50% glucuronic acid, and 0 to 50% iduronic acid, in terms of the relative molar percentage of monomers. Preferably, the ulvan comprises 15% to 50% glucuronic acid, and 0 to 30% iduronic acid, in terms of the relative molar percentage of monomers. More preferably, the ulvan comprises 25% to 50% glucuronic acid, and 0 to 15% iduronic acid, in terms of the relative molar percentage of monomers.

In some embodiments, the ulvan comprises 25% to 100% of the disaccharide unit [glucuronic acid-rhamnose sulphate] and 25% to 100% of the disaccharide unit [iduronic acid-rhamnose sulphate], in terms of the relative molar percentage of units. Preferably, the ulvan comprises 40% to 100% of the disaccharide unit [glucuronic acid-rhamnose sulphate] and 25% to 70% of the disaccharide unit [iduronic acid-rhamnose sulphate], in terms of the relative molar percentage of units. More preferably, the ulvan comprises 40% to 100% of the disaccharide unit [glucuronic acid-rhamnose sulphate] and 25% to 50% of the disaccharide unit [iduronic acid-rhamnose sulphate], in terms of the relative molar percentage of units.

In particular embodiments, ulvan comprises 25% to 100% of the A3S and 25% to 100% of the B3S repeating disaccharide unit, in terms of the relative molar percentage of units. Preferably, the ulvan comprises 40% to 100% of the A3S and 25% to 70% of the B3S repeating disaccharide unit, in terms of the relative molar percentage of units. More preferably, the ulvan comprises 40% to 100% of the A3S and 25% to 50% of the B3S repeating disaccharide unit, in terms of the relative molar percentage of units.

Ulvan is soluble in aqueous solutions, and is insoluble in almost all organic solvents. Ulvan molecule surface charge depends on solution pH and ionic strength. Changes in solution osmolality and pH, and in ulvan concentration, result in solutions with different viscosity. Ulvan has the ability to form weak gels in the presence of divalent cations, such as boron and calcium (Lahaye et al., 2007).

In the context of this invention, ulvan functional groups, such as hydroxyl, carboxyl and sulphate groups, can be used to incorporate additional functional groups like methacrylate, amine, aldehydes, itaconates, acrylamides, or acrylates. In this regard, the degree of substitution of the ulvan functional groups may be between 0.1% and 50%. In a preferred embodiment, the degree of substitution of the ulvan functional groups is between 0.1% and 40%, preferably below 30%.

The composition is preferably in liquid form. Preferably, the composition is an aqueous composition. Preferably, the ulvan has a concentration of 0.1% to 30% w/V in the composition. In some embodiments, the ulvan has a concentration of 0.2% to 20% w/V in the composition. In other embodiments, the ulvan has a concentration of 0.3% to 15% w/V in the composition. In particular embodiments, the ulvan has a concentration of 0.4% to 12% w/V in the composition. In certain embodiments, the ulvan has a concentration of 0.5% to 10% w/V in the composition.

The composition may comprise a buffer to help to maintain the composition at a particular pH. The buffer may be any suitable buffer, for example, a phosphate buffer or a Tris-HCl buffer.

The composition may have a pH of 4 to 10. In some embodiments, the composition has a pH of 5 to 9. In other embodiments, the composition has a pH of 6 to 8. In further embodiments, the composition has a pH of 6.8 to 7.4. In particular embodiments, the composition has a pH of about 7.

In some embodiments, the composition comprises physiological saline solution or cell culture media.

The composition may comprise one or more sulphated polysaccharides in addition to the ulvan. Preferably, the sulphated polysaccharides are selected from the group consisting of gellan sulphate, chondroitin sulphate, keratan sulphate, heparin sulphate, dextran sulphate and xylose sulphate. These are selected according to the following order of preference (starting with the most preferred): chondroitin sulphate, gellan sulphate, xylose sulphate, dextran sulphate, keratan sulphate, and heparin sulphate.

In some embodiments, the composition further comprises hyaluronic acid.

In some embodiments, the composition is totally or partially in acid form. Alternatively, the composition (in particular the acid groups) may be totally or partially in salt form. The salt should be a physiologically acceptable salt. The salt may be formed with an alkali or alkaline earth metal, such as Na⁺, K⁺, Sr⁺, Ca²⁺, Ba²⁺, Mg²⁺, or with organic compounds such as primary amines, secondary amines or tertiary amines.

The composition may comprise 0.1% to 10% w/V of a salt. In some embodiments, the composition comprises 0.2% to 8% w/V of a salt. In other embodiments, the composition comprises 0.3% to 7% w/V of a salt. In various embodiments, the composition comprises 0.4% to 6% w/V of a salt. In particular embodiments, the composition comprises 0.5% to 5% w/V of a salt. The salt may be any suitable salt as suggested above. In some embodiments, the salt is NaCl.

In various embodiments, the composition comprises additional salts such as CuSO₄ and/or H₃BO₄.

The compositions described above can be used in the preparation of pharmaceutical compositions to be used in the treatment and prophylaxis of osteoarthritis. The compositions can be combined with other excipients or active substances used in the context of veterinarian and human medicine.

The compositions can be administered by various routes, including topical, enteral and parenteral. Parenteral administration routes include intra-arterial, intra-articular, intracavitary, intradermal, intralympathic, intramuscular, intrasynovial, intravenous, and subcutaneous. Enteral routes include oral and gastro-intestinal. Topical routes include application into the skin and mucous membranes.

In a preferred embodiment, the composition is delivered to a patient by intra-articular injection into a diseased joint, which can be repeated according to a clinical prescription regime. In an even more preferred embodiment, the composition is delivered by a single intra-articular injection into a diseased joint.

Dosage of the composition can be adapted to the administration route, as well as to the patient profile, including age, gender, condition, disease progression, or any other phenotypic or environmental parameters.

The composition may be in a solid form such as an amorphous, crystalline or semi-crystalline powder, granules, flakes, pills, scaffolds, capsules and suppositories. Such a solid form can be converted into a liquid form by mixing the solid with a physiologically appropriate liquid such as solvents, solutions, suspensions and emulsions.

In another aspect, the present invention provides a method of treating a patient suffering from a musculoskeletal disease, the method comprising administering an effective amount of the composition described above to the patient.

As described above, the composition may be administered by intra-articular injection, for example, into a joint of the patient. The joint may be an arthritic joint.

The musculoskeletal disease may be arthritis. The arthritis may be osteoarthritis arthritis (arthrosis) or rheumatoid arthritis. In some embodiments, the patient is suffering from rheumatoid arthritis. In other embodiments, the patient is suffering from osteoarthritis.

In a further aspect, the present invention provides ulvan for use in the treatment or prophylaxis of a musculoskeletal disease, for example, arthritis. Further, the present invention provides the use of ulvan in the manufacture of a medicament for the treatment or prophylaxis of a musculoskeletal disease, for example, arthritis.

In a particular aspect, the invention provides the composition described above for use in therapy. Further, the present invention provides the composition described above for use in the treatment or prophylaxis of a musculoskeletal disease, for example, arthritis. In addition, the present invention provides the use of the composition described above in the manufacture of a medicament for the treatment or prophylaxis of a musculoskeletal disease, for example, arthritis.

### BRIEF DESCRIPTION OF THE FIGURES

The invention will now be described in detail, by way of example only, with reference to the following Figures:

FIG. 1. Schematic representation of four possible repetition units of ulvan: two aldobiouronic acids (A3S and B3S), and two ulvanobioses (U3S and U2S3S).

FIG. 2. ¹H NMR spectrum at 60° C. of ulvan dissolved in deuterium oxide.

FIG. 3. ¹H NMR spectrum at 60° C. of glucuronic acid dissolved in deuterium oxide.

FIG. 4. ¹H NMR spectrum at 60° C. of rhamnose dissolved in deuterium oxide.

FIG. 5. ¹H NMR spectrum at 60° C. of xylose dissolved in deuterium oxide.

FIG. 6. ¹H NMR spectrum at 70° C. of methacrylated ulvan dissolved in deuterium oxide. Arrow indicates the peaks of new protons in ulvan backbone which results from methacrylation reaction.

FIG. 7. Image of the hydrogels produced from methacrylated ulvan after 10 minutes UV-light irradiation. Left: front image of the hydrogel; Right: top image of the hydrogel.

FIG. 8. Gene expression ratio of collagen type II, collagen type I and collagen type X after treatment, normalized to non-treated group.

FIG. 9. Microscopic imaging (5×) of rabbit articular cartilage sections stained with safranin O. Left: Staining of normal articular cartilage and subchondral bone. Middle: Staining of articular cartilage after 3 weeks of osteoarthritis (OA) induction. Right: Staining of osteoarthritic articular cartilage after 8 weeks of treatment with ulvan formulation (3% w/V).

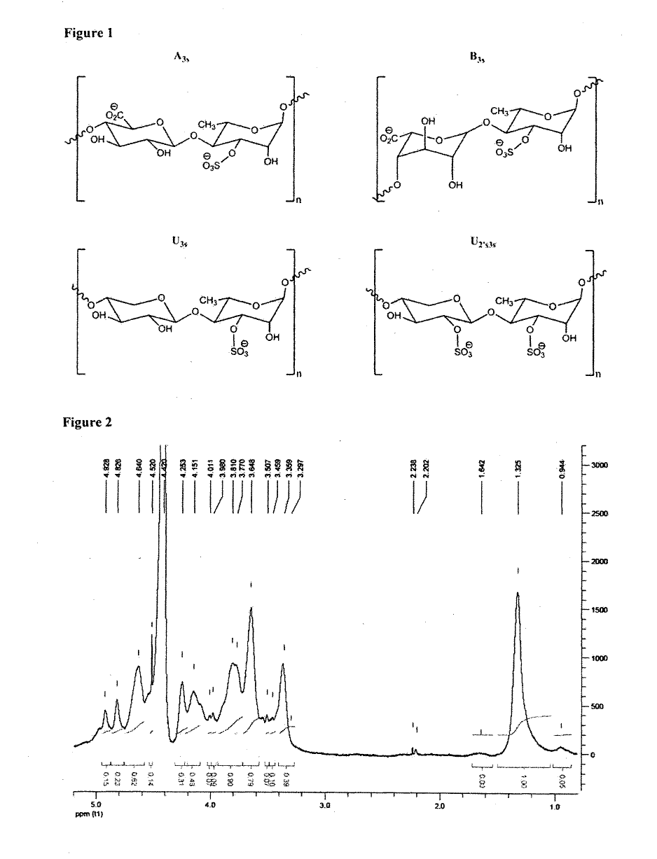

### DETAILED DESCRIPTION OF THE INVENTION

The following examples are merely illustrative and should not be construed to limit the scope of the disclosure.

### Example 1: Characterization of Ulvan Salts

Ulvan salt was prepared in different solvents for chemical characterization.

### Methods & Results

### Monomeric Composition

Ulvan was solubilized in H₂O at room temperature, and then hydrolysed with H₂SO₄ 1 M at 100° C. for 2.5 hours. Neutral sugars were determined as alditol acetates using gas chromatography analysis as described by Coimbra et al. (1996). Uronic acids were determined by an adapted 3-phenylphenol colorimetric method described by Coimbra et al. (1996). Linkage analysis was executed by methylation as adapted by Coimbra et al. (1996). Results are shown in Table 1.

### Repetition Unit and Sulphate Content

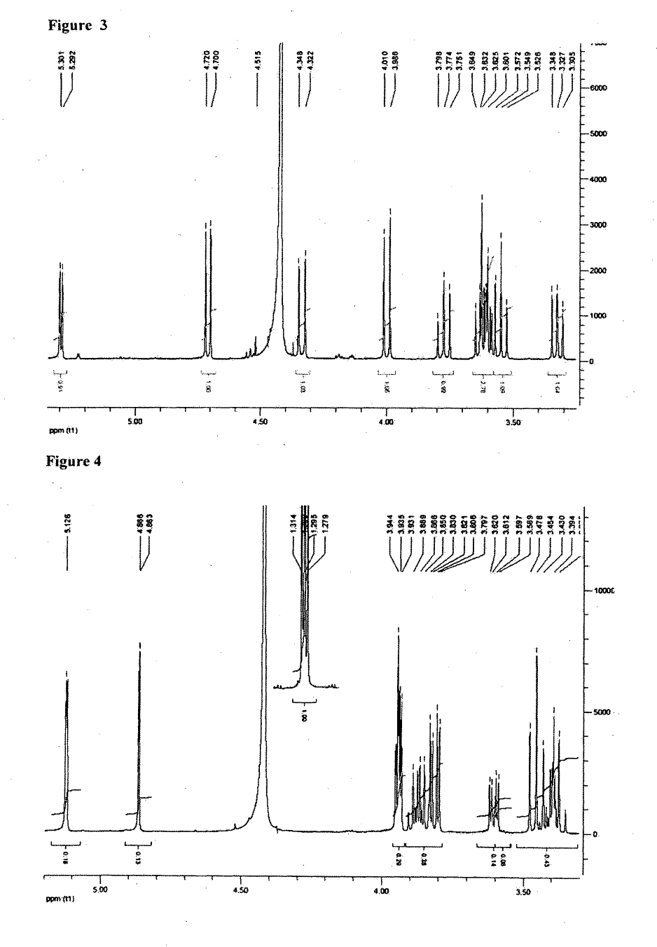

Ulvan (1% w/V) was solubilized in deuterium oxide at room temperature. Sample was analysed by ¹H NMR acquired on a Varian Unity Plus (Varian, USA) spectrometer at 60° C. (FIG. 2).

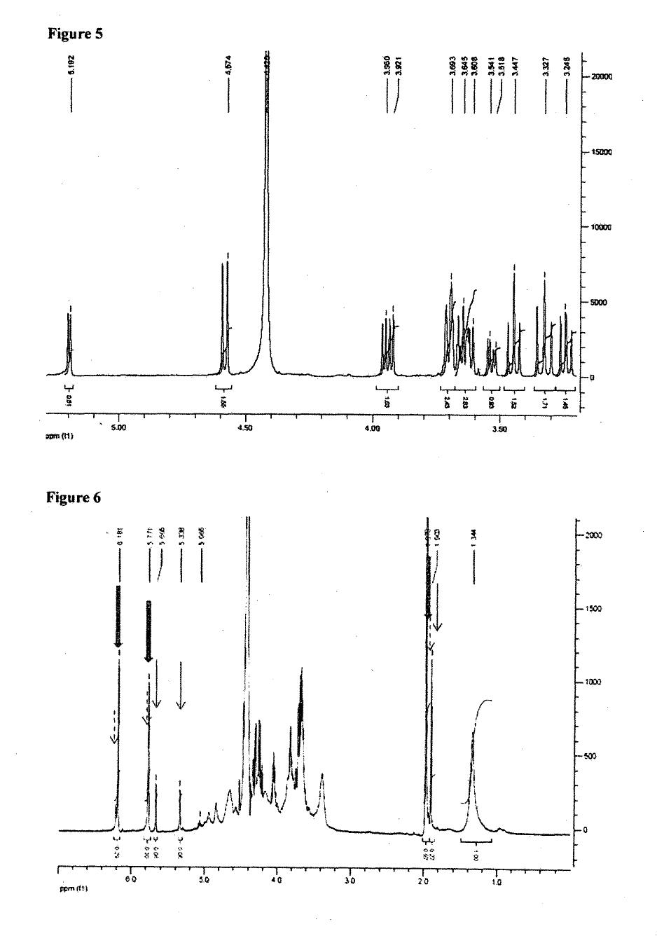

¹H-NMR spectra were also obtained for the monomers mostly present within ulvan composition (glucuronic acid, rhamnose and xylose, FIGS. 3-5). These monomers were prepared at 1% w/V solution in deuterium oxide, as for the ulvan solution. The chemical shifts of the main repetition unit of the produced ulvan were identified based on the literature (Barros et al., 2013; Lahaye, Inizan, & Vigouroux, 1998; Robic, Sassi, & Lahaye, 2008). The intensity of rharnose and glucuronic acid peaks was compared with the remaining peaks of the spectra, in order to identify the relative percentage of the repetition unit, such as the aldobiouroninc A3S (>4-beta-D-glucuronic acid-(1>4)-alpha-L-rhamnose 3-sulphate-1>). By the relative composition of monomers and analysis of their linkage, the relative composition of aldobiouronic and ulvanobiose repetition unit can also be determined. Results are shown in Table 1.

Sulphate content of ulvan sample was calculated based on ¹H NMR spectrum analyses assuming that: (i) all the rhamnose units are sulphated; and (ii) there exists one unit of rhamnose per glucuronic acid. Considering these assumptions, the calculation is made by the following developed Equation 1. Results are in Table 1.

$$\% S = \frac{{w}_{S}}{{w}_{total}} \times 100 = \frac{\frac{{Mw}_{S} \times {A}_{RhmS}}{{Mw}_{RhmS}} \times {Mw}_{S}}{{Mw}_{RhmS} \times {A}_{RhmS} + {Mw}_{GA} \times {A}_{GA}} \times 100 = \frac{26.35 \times {A}_{RhmS}}{243.22 \times {A}_{RhmS} + 194.14 \times {A}_{GA}} \times 100 \qquad$$

Equation 1—Equation developed to calculate ulvan sulphate percentage using ¹H NMR spectrum. Where wₛ: sulfur weight; Wₜₒₜₐₗ: total weight; Mw: molecular weight; A:anomeric C peak integration: RhmS: sulphated rhamnose; GA: glucuronic acid; S:sulfur

### Estimated Sulphate Degree

Based on sulphate content and molecular weight of ulvan, the sulphation degree was estimated using equation 2. Results are in Table 1.

$${S}_{T} = \frac{Mw \times S \%}{100}$$

$${n}_{S} = \frac{{S}_{T}}{{Mw}_{S}}$$

$${O}_{T} = {n}_{S} \times 3 \times {Mw}_{O}$$

$${OH}_{T} = ( \frac{Mw - {S}_{T} - {O}_{T}}{{Mw}_{A}} ) \times 4$$

$$SD = ( \frac{{n}_{S}}{{OH}_{T}} ) \times 100$$

Equation 2—Equation developed to estimate ulvan sulphation degree (SD). Where S_{T}: Total sulfur mass in the molecule; Mw: molecular weight of ulvan; S %: sulphate content of ulvan, n_{S}: estimated number of sulfur atoms; Mw_{S}: Sulfur molecular weight; O_{T}: Total oxygen mass in the molecule; Mw_{O}: Oxygen molecular weight; OH_{T}: Total number of hydroxyl groups in the molecule; Mw_{A}: Molecular weight of a repeating unit without sulfur; Note that molecular weight of A3S and B3S repeating units are equivalent, equation applies equally to both.

### Molecular Weight

Ulvan was solubilized in NaCl 0.3 M (eluent solution) at a final concentration of 0.1% w/V. The solution was analysed by gel permeation chromatography in the equipment Viscotek TDA 305, with the three detectors: light scattering, refractive index and viscometer. Column set was composed by a guard pre-column Aq. Guard (Viscotek) and a PLaquagel-OH Mixed 8 μm (Polymer Laboratories). Elution was performed at 30° C. using a flow rate of 1 ml/min. Triple detection calibration using polyethylene oxide as narrow standard was performed for molecular weight (Mw) calculation. Results are in Table 1.

TABLE 1
Chemical characterization of ulvan samples

| | Monosaccharide (mol %) | | | Aldobiouronic | | | |
|---|---|---|---|---|---|---|---|
| Ulvan | | Glucuronic | | unit (A3S and | Sulphate | Mw | |
| sample | Rhamnose | acid | Xylose | B3S) (%) | (%) | (KDa) | SD |
| Ulvan | — | — | — | — | 4.60 ± 0.11 | 1045 ± 102 | 14.5% |
| CRD | | | | | | | |
| Ulvan | 29.1 ± 0.3 | 23.2 ± 0.1 | 33.6 ± | ≈75 | 4.99 ± 0.37 | 639 ± 108 | 15.9% |
| PRFD | | | 0.8 | | | | |
| Ulvan | — | — | — | ≈90 | 5.00 ± 0.15 | 266 ± 19 | 16.0% |
| 121314 | | | | | | | |

Ulvan samples obtained from different processes were characterized for its chemical properties. Notorious differences were registered between ulvan samples, especially on the average molecular weight (Mw). Additionally, the composition of the tested samples is also remarkably distinct, whereas aldobiouronic units were the most present in analysed samples. This fact indicates that ulvan 121314, with 90% aldobiouroninc, possibly A3S by ¹H NMR analysis, is expected to present less than 5 mol % of the monosaccharide xylose, whereas the ulvan PRFD presents almost 30 mol %.

### Example 2: Degradation of Ulvan by Cartilage Degradative Enzymes

Osteoarthritic joints are characterized for expression of enzymes that degrade the cartilage matrix components, namely hyaluronidase. Additionally, hyaluronidase promotes rapid degradation of hyaluronic acid based viscosupplements accelerating its clearance.

### Methods

### Resistance to Hyaluronidase

Ulvan was dissolved in phosphate buffer saline (PBS) at room temperature at the final concentrations of 0.5, 1.0 and 3.0% w/V. Hyaluronic acid (HA) was dissolved in phosphate buffer saline at room temperature for the final concentrations of 0.1, 0.5 and 1.0% w/V. Solutions were incubated in optimal conditions (pH 7, 37° C., 48 hours, 75 rpm) with hyaluronidase (EC 3.2.1.35). Solutions were then evaluated for polysaccharide molecular weight alteration by gel permeation chromatography as described above. Results are shown in Table 2.

### Results

TABLE 2
Molecular weight of ulvan and hyaluronic acid after 48 hours
of incubation with hyaluronidase in optimal conditions.
Samples incubated without enzyme were used as control.

| Sample | Hyaluronidase (units/mL) | Mw (KDa) |
|---|---|---|
| Ulvan 121314 | 0 | 266 ± 19 |
| | 300 | 278 ± 12 |
| Hyaluronic acid | 0 | 589 ± 15 |
| | 300 | 27 ± 17 |

HA molecular weight was reduced by 97.3±1% by incubation with hyaluronidase, although ulvan had no molecular weight alteration. These results demonstrate that ulvan is resistant to hyaluronidase degradation in optimal conditions and hyaluronidase action over HA is dramatic. Given that HA viscosity is described in the literature as being associated with its high molecular weight, the degradation power of hyaluronidase over HA is likely to reduce HA efficiency as a viscosupplement. The greater stability of ulvan towards hyaluronidase means that ulvan will remain intact over a longer timeframe than hyaluronic acid and as a consequence, lower dosages and fewer injections of ulvan should be required for effective pain relief.

### Example 3: Anti-Oxidant and Anti-Coagulant Potential of Ulvan

Osteoarthritic joints present an oxidative environment, which is associated with inflammation and pain. Additionally, parenteral administration of a viscosupplement, for example by intra-articular injection, presents high risk of bloodstream contact, for which it is important to avoid potential induction of blood clot formation. This example demonstrates the reducing power capacity of ulvan and proves that ulvan is non-thrombogenic, which is relevant for its potential application as an injectable formulation.

### Methods

Ulvan samples and hyaluronic acid were dissolved at 0.5% w/V in the mandatory solvent for each analysis performed. Hyaluronic acid is the gold standard for osteoarthritis therapy, therefore it was used in this example as a basis for comparison. Heparin is a well-known standard for anti-coagulant activity tests. The reducing power of samples was quantified by the following protocol. Samples were prepared in PBS and mixed with potassium ferricyanide, then heated at 50° C. for 20 min. The reaction was stopped by the addition of trichloroacetic acid solution (10% w/V). The solution was centrifuged and supernatant mixed with distilled water and ferric chloride (0.1% w/V). Absorbance was measured at 700 nm. Ascorbic acid was used as standard for reducing power. Results are in Table 3.

Anti-coagulant activity was quantified using heparin as a reference substance. Measurement of prothrombin time (PT) and activated partial thrombiplastin time (aTPP) was performed as previously described by Subhapradha et al. Results are shown in Table 4.

### Results

TABLE 3
Reducing power capacity of ulvan samples and hyaluronic
acid with ascorbic acid as standard

| Sample (0.5% w/V) | Reducing power % |
|---|---|
| Ulvan 121314 | 35 ± 4 |
| Hyalufonic acid | 3 ± 0 |

Ulvan presented 35% reducing power compared to ascorbic acid. Hyaluronic acid tested under the same conditions did not provide significant reducing power. This result indicates that ulvan can act as a potential anti-oxidant agent in osteoarticular environments unlike hyaluronic acid.

TABLE 4
Anti-coagulant activity of ulvan
evaluated by PT and aPTT methods

| Blood treatment | PT (s) | aPTT (s) |
|---|---|---|
| None | 10.0 | 22.5 |
| 0.5% w/V ulvan in PBS | 10.6 | 81.1 |
| 0.05% w/V heparin in PBS | 14.4 | 364.6 |

Ulvan presents anti-coagulant activity, by slightly increasing the coagulation time in PT test and very significantly in aPTT test. When compared to heparin, it is possible to verify that ulvan's anti-coagulant activity is not as high as heparin. This result indicates that ulvan will not promote an ischemic response, which is highly relevant taking into account its possible administration by parenteral routes.

These results indicate that ulvan can act as anti-oxidant agent in osteoarticular environments without eliciting a thrombogenic response.

### Example 4: Chemical Modification of Ulvan

Every ulvan repetition unit presents at least one reactive group: hydroxyl, carboxyl and sulphated group. These available groups allow further chemical modification of ulvan polysaccharide.

### Methods

Methacrylated ulvan was manufactured by application of the method described in WO 2011/119059 A1. Methacrylated ulvan, the reaction product, was solubilized in deuterium oxide at room temperature. The sample was analysed by ¹H NMR acquired on a Varian Unity Plus (Varian, USA) spectrometer at 70° C. Substitution degree was calculated based on the method from M. Hamcerencu M. (2008).

Methacrylated ulvan was dissolved in Tris-HCl 2 M and H₃BO₄ 40 mM for final concentration of 4% w/V. Photo-initiator methyl benzoylformate (MBF) was added. The solution was exposed to UV-light and methacrylated ulvan hydrogels were produced.

### Results

FIG. 6 represents a ¹H NMR spectrum at 70° C. of reaction product dissolved in deuterium oxide. The signals in the spectral region of 5.33-6.18 ppm were attributed to the vinyl carbon-linked hydrogen (C═CH2). The peaks shown at 1.90-1.97 ppm, were ascribed to methyl groups adjacent to the double bond (CH3-C═CH2). These signals indicate the successful formation of methacrylated ulvan. Methacrylated ulvan substitution degree was 17%±1.

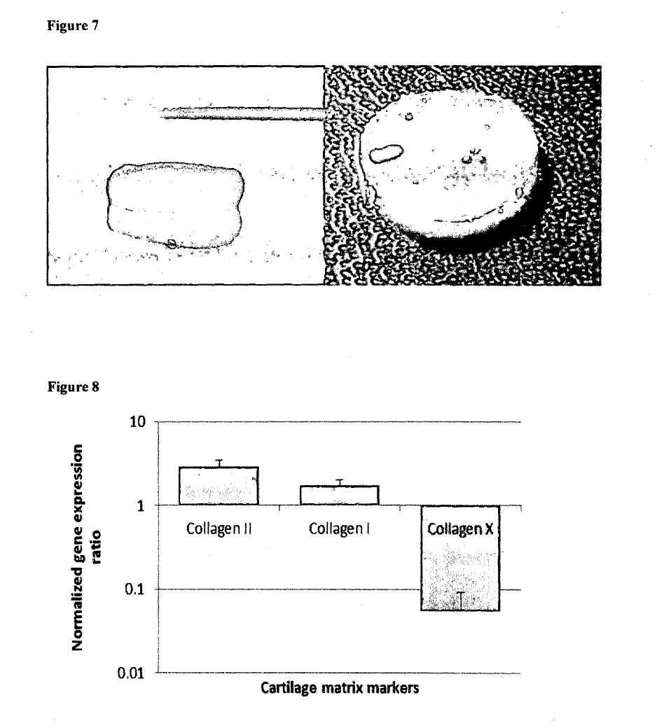

FIG. 7 illustrates methacrylated ulvan hydrogels produced by photo-crosslinking. Hydrogels present the shape of the used mold and a yellowish coloration, which is typical of ulvan solutions.

### Example 5: Evaluation of the In Vitro Effect of Ulvan Formulations on a Primary Culture of Human Osteoarthritic Articular Chondrocytes

This procedure may be applied to the evaluation of any ulvan formulation according to the invention.

### Methods

Human articular cartilage was obtained from patients diagnosed with osteoarthritis and undergoing total knee replacement. Chondrocytes were isolated and cultured following methods described by Masuda and Sah (2006). Osteoarthritic chondrocytes were exposed to ulvan formulation (Z014, see table 5) (treated group), or culture media only (untreated group) for 24 h. Cells were further collected for gene expression of collagen type II, collagen type I and collagen type X, by quantitative real time polymerase chain reaction (qRT-PCR). Gene expression of treated group was normalised to untreated control group, and presented as normalised expression ratio, according to Livak and Schmittgen (2011). Data is presented as average±SD.

### Results

FIG. 8 represents the normalised expression ratio of three genes quantified by real time PCR (qRT-PCR)-genes coding for collagen type II, collagen type I and collagen type X proteins. After treatment, ulvan formulation induced a 2.9× increase in the expression of collagen type II, relatively to untreated cells, marker of healthy cartilage matrix. Regarding expression of proteins related to unhealthy cartilage matrix, while collagen type I was upregulated 1.7×, collagen type X was downregulated 0.06× relatively to untreated cells. This trend indicates that exposure of OA chondrocytes to ulvan formulation most notably promotes gene expression related with collagen type II expression, which is a sign of healthy extracellular expression.

### Example 6: Evaluation of the In Vivo Effect of Ulvan Formulations on Rabbit Osteoarthritic Articular Cartilage Model

This procedure may be applied to the evaluation of any ulvan formulation according to this invention.

### Materials & Methods

A rabbit osteoarthritis model was used to test the effects of ulvan formulations (A132, see table 5) on the progression of OA. An osteoarthritic condition was induced in the animal's knee by medial meniscectomy following methods described by Smith and Little (2007). An 8 week treatment was tested by periodic intra-articular injection of ulvan formulation every 2 weeks. After treatment, articular cartilage samples were harvested for histological analysis. Safranin O/fast green staining was performed to identify status of cartilage matrix.

### Results

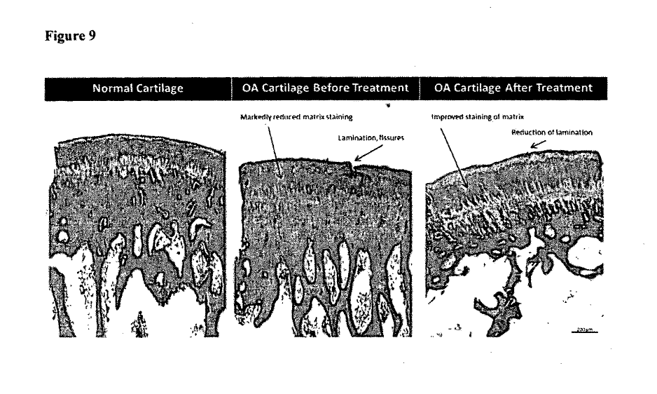

FIG. 9 represents microscopic images of rabbit articular cartilage sections stained with safranin O/fast green. The left image presents staining of normal articular cartilage (stained red) and subchondral bone (stained blue-green). The middle image demonstrates staining of articular cartilage immediately after osteoarthritis induction. Signs of OA are revealed such as delamination and fibrillation of the cartilage surface, fissures within the matrix as well as glycosaminoglycan loss within the tissue. The right image presents staining of osteoarthritic articular cartilage after 8 weeks of treatment with ulvan formulation (3% w/V). Visible improvement of the physical condition of the articular cartilage is observed: stronger staining of the cartilage matrix as well as lower fibrillation and fissures.

### Example 7—Ulvan Formulations

Ulvan formulations are prepared under aseptic environment. Ulvan in powder form can be dissolved in an appropriate liquid. Table 5 presents ulvan formulations and their composition.

### Preparation of Ulvan Formulation A132

Under aseptic environment, 30 mg of ulvan in solid form is dissolved in 1 mL of sterile phosphate buffer saline, pH 7. Dissolution occurs at room temperature in 30 minutes under rotational agitation.

TABLE 5
Composition of ulvan formulations

| | Ulvan | | |
|---|---|---|---|
| Ulvan | concentration | | Additional |
| formulation ID | (% w/V) | Liquid | components |
| A125 | 0.5 | phosphate | None |
| A128 | 1.0 | buffered | |
| A132 | 3.0 | saline, pH 7 | |
| A138 | 10 | | |
| B125 | 0.5 | phosphate | Hyaluronic |
| B128 | 1.0 | buffered | acid |
| B132 | 3.0 | saline, pH 7 | |
| B138 | 10 | | |
| E125 | 0.5 | 0.9% w/V | None |
| E128 | 1.0 | NaCl, pH 7 | |
| E132 | 3.0 | | |
| E138 | 10 | | |
| E155 | 0.5 | 0.9% w/V | Hyaluronic |
| E159 | 1.0 | NaCl, pH 7 | acid |
| E163 | 3.0 | | |
| E175 | 10 | | |
| E202 | 0.5 | Tris-HCl buffer, | none |
| E203 | 1.0 | pH 7 with CuSO₄ | |
| E205 | 3.0 | and H₃BO₄ | |
| E212 | 10 | | |
| Z014 | 3.0 | Cell culture media | None |

### REFERENCES

Alves, A., Caridade, S. G., Mano, J. F., Sousa, R. A. & Reis, R. L. (2010) Extraction and physico-chemical characterization of a versatile biodegradable polysaccharide obtained from green algae. Carbohydrate Research 345: 2194-2200. doi: 10.1016/j.carres.2010.07.039
Barros, A. A. A., Alves, A., Nunes, C., Coimbra, M. A., Pires, R. A. & Reis, R. L. (2013). Carboxymethylation of ulvan and chitosan and their use as polymeric components of bone cements. Acta Biomaterialia, (July), 1-12. doi:10.1016/j.actbio.2013.06.036
Coimbra, M. A., Delgadillo, I., Waldron, K. W. & Selvendran, R. R. (1996). Isolation and Analysis of Cell Wall Polymers from Olive Pulp. In H.-F. Linskens & J. F. Jackson. Modern Methods of Plant Analysis (pp. 19-44). Berlin: Springer-Verlag. Lahaye, M., Inizan, F. & Vigouroux, J. (1998). NMR analysis of the chemical structure of ulvan and of ulvan-boron complex formation. Carbohydrate Polymers, 36, 239-249.
Robic, A., Sassi, J.-F. & Lahaye, M. (2008). Impact of stabilization treatments of the green seaweed Ulva rotundata (Chlorophyta) on the extraction yield, the physico-chemical and rheological properties of ulvan. Carbohydrate Polymers, 74(3), 344-352. doi:10.1016/j.carbpol.2008.02.020
Gonalves, C., Rodriguez-Jasso, R. M., Gomes, N., Teixeira, J. A. & Belo, I. (2010). Adaptation of dinitrosalicylic acid method to microtiter plates. Analytical Methods, 2(12), 2046
Subhapradha N., Suman S., Ramasamy P., Saravanan R., Shanmugan V., Srinivasan A. & Shanmugan A. (2013) Anticoagulant and antioxidant activity of sulphated chitosan from the shell of donacid clam Donax scortum (Linnaeus, 1758); International Journal of Nutrition, Pharmacology, Neurological Diseases. 3(1):39-45. Hamcerencu, M., Desbrieres, J., Khoukh A., Popa M. & Riess G. (2008) Synthesis and characterization of new unsaturated esters of gellan gum. Carbohydrate Polymers, 71, 92-100.
Masuda, K & Sah R L in “Culture of cells for tissue engineering”, Ed.: Vunjak-Novakovic G and Freshney R I, 2006
Livak, K. J. & Schmittgen T. D., Methods 25, 402-408, 2011
Smith M. M. & Little C. B. in “Osteoarthritis” Ed.: Moskowitz et al., Wolters Kluver, 2007.
Lahaye, M. & Robic, A. (2007) Structure and functiontional properties of ulvan, a polysaccharide from green seaweeds. Biomacromolecules, 8(6), 1765-1774.
Papy-Garcia, D., Barbier-Chassefiere, V., Rouet, V., Kerros, M-E., Klochendler, C., Tournaire, M-C., Barritault, D., Caruelle, J-P. & Petit, E. (2005) Nondegradative sulfation of polysaccharides. Synthesis and structure characterisation of biologically active heparin sulphate mimetics. Macromolecules, 38, 4647-4654.
Nagasawa, K., Inoue, Y. & Kamata, T. (1977) Solvolytic desulfation of glycosamihoglycutonan sulfates with dimethyl sulfoxide containing water or methanol. Carbohydrate Research, 58, 47-55.
Suzuki, K., Otani, J. & Tanaka, S. (2009) Angiogenesis inhibitor and use thereof. JP2009057285 (Konan Chemical Manufacturing C°, LTD)

1. A composition comprising ulvan for the treatment or prophylaxis of arthritis.

2. The composition of claim 1, wherein the sulphation degree of ulvan is 0.1% to 75%.

3. (canceled)

4. The composition of claim 1, wherein the sulphation degree of ulvan is 0.1% to 6%.

5. The composition of claim 1, wherein the ulvan has an average molecular weight between 100 and 10,000 kDa.

6. (canceled)

7. The composition of claim 1, wherein the ulvan has an average molecular weight between 100 and 600 kDa.

8. The composition of claim 1, wherein the ulvan comprises 5% to 50% glucuronic acid, and 0 to 50% iduronic acid, in terms of the relative molar percentage of monomers.

9. (canceled)

10. The composition of claim 1, wherein the ulvan comprises 25% to 100% of a first disaccharide unit [glucuronic acid-rhamnose sulphate] and 25% to 100% of a second disaccharide unit [iduronic acid-rhamnose sulphate], in terms of the relative molar percentage of units.

11. (canceled)

12. The composition of claim 1, wherein the ulvan comprises 25% to 100% of the A3S and 25% to 100% of the B3S repeating disaccharide unit, in terms of the relative molar percentage of units.

13. (canceled)

14. (canceled)

15. (canceled)

16. (canceled)

17. The composition of claim 1, wherein the ulvan has a concentration of 0.1% to 30% w/V in the composition.

18. (canceled)

19. (canceled)

20. (canceled)

21. (canceled)

22. (canceled)

23. The composition of claim 1, further comprising one or more sulphated polysaccharides in addition to the ulvan.

24. The composition of claim 23, wherein the one or more sulphated polysaccharides are selected from the group consisting of gellan sulphate, chondroitin sulphate, keratan sulphate, heparin sulphate, dextran sulphate and xylose sulphate.

25. The composition of claim 1, further comprising hyaluronic acid.

26. (canceled)

27. (canceled)

28. (canceled)

29. The composition of claim 1, for use in therapy.

30. The composition of claim 1, for use in the treatment or prophylaxis of a musculoskeletal disease including arthritis.

31. Ulvan for use in the treatment or prophylaxis of a musculoskeletal disease including arthritis.

32. A method of treating a patient suffering from a musculoskeletal disease, the method comprising administering an effective amount of the composition of claim 1 to the patient.

33. The method of claim 32, wherein the composition is administered by intra-articular injection into a joint of the patient.
